(19) United States
(12) Patent Application Publication
(10) Pub. No.: US 2002/0071836 A1
(43) Pub. Date: Jun. 13, 2002

(54) METHOD OF STIMULATING THE EXPRESSION OF CYTOKINES IN MAMMALS USING SUGAR-PROCESSINIG ENZYMES AND NUTRITIONAL SUPPLEMENTS CONTAINING SUCH ENZYMES

(76) Inventors: JOACHIM LEHMANN, SCOTTSDALE, AZ (US); AFTAB J. AHMED, SCOTTSDALE, AZ (US); RUDOLF KUNZE, BERLIN (DE)

Correspondence Address: GEORGE A. LOUD, 3137 MOUNT VERNON AVENUE, ALEXANDRIA, VA, 22305, US

(21) Appl. No.: 09/323,247

(22) Filed: Jun. 10, 1999

(51) Int. Cl. A61K 38/47

(52) U.S. Cl. 424/94.61

(57) ABSTRACT

Sugar-processing enzymes are used in a method of stimulating the expression of anti-inflammatory cytokines by mammalian peripheral blood cells and are incorporated into nutritional supplements. The preferred sugar-processing enzymes are cellulases and glucolytic enzymes.

### FIELD OF INVENTION

This invention relates to the use of sugar-processing enzymes, including glucolytic enzymes and cellulases to induce the expression of anti-inflammatory cytokines, and to the use of such enzymes as immune-stimulatory agents in the mammals. The invention also relates to nutritional supplements containing sugar-processing enzymes.

### BACKGROUND OF THE INVENTION

Certain protein hormones called cytokines are important modulators of both the animal and human immune systems. Cytokines are produced in the body by various immune-competent cells, e.g. mammalian peripheral white blood cells, and are characterized by their distinct structures and functions. Cytokines are critical to host defense against pathogens and enable small numbers of lymphocytes specific for any one antigen to activate a variety of effector mechanisms to eliminate the antigen. They play an important role in a variety of processes to combat cancer, and bacterial and viral infections, along with their involvement in autoimmune diseases such as rheumatic disorders. Thus, stimulation of the expression of cytokines by the mammalian immune system is considered to reduce likelihood of such diseases and to enhance health in general.

An array of stimuli induce cytokines. Thus, among the endogenous mediators of cytokine induction are interleukins, circulating immune complexes and proteins/factors of the complement cascade. Likewise, exogenous factors, such as lectins, glucans, microbial antigens and endotoxins also induce cytokines. Some of these mediators, including recombinant interleukins, glucans, several microbial agents and plant lectins, have therapeutic value. Given their immense cost, however, the mediators do not lend themselves to use as therapeutic agents. Their use as therapeutic agents is directed to stimulate the immune network in the body and, thus, to enhance health. Most of these agents function both in vitro and in vivo to induce cytokines.

Interleukin-10 and -12, in particular, are important cytokines. Interleukin-10 (IL-10) is produced by T-cells, B-cells, macrophages and keratinocytes, and enhances B-cell proliferation and attendant antibody secretion. IL-10 deactivates activated monocytes and T-cells by reducing the synthesis of proinflammatory cytokines. Thus, IL-10 plays an indirect role in the differentiation of T-lymphocytes and, as such, augments the humoral immune response in the body.

Interleukin-12, on the other hand, is produced in macrophages and B-cells and plays an important regulatory role in differentiation of T-lymphocytes and natural killer cells. IL-12 enhances cell-mediated immunity, and, therefore, predisposes the body to a better defense against viral infections and intracellular effectors. Therefore, it is important to identify effective and cost-effective inducers of cytokines, but particularly the inducers of regulatory cytokines IL-10 and IL-12.

Generally, the term cellulase refers to undefined extracellular mixtures produced by various fungi, bacteria, insects and lower animals and to enzymes isolated therefrom, that hydrolyze cellulose. Typical fungi secreting cellulase are Trichoderma reesei, T. Viride, Aspergillus niger and Penicillium funiculosum. The cellulase produced by the fungi mainly consists of three types of enzymes: Endoglucanase, exoglucanase and β-glucosidase. In addition to the hydrolytic activities, transglycosylation by cellulase is well known. Recently a cellulase-catalyzed lactosylation of alkyl cellobioside giving rise to cello-oligosaccharide derivatives in a mixed buffer/organic solvent system has been reported. Although translactosylation and transgluosylation among transglycosylation have been reported in numerous reports, it is only recently that a transgalactosylation to catalyze galatosyl glycoside synthesis, such as galacto-oligosacchardide (GOS), has been reported. In addition, the use of cellulases in the synthesis of ethanol as an alternative to fossilized fuel has been extensively documented.

### SUMMARY OF THE INVENTION

Accordingly, it is an object of the present invention to provide novel and economical stimulators and immunomodulators of the mammalian immune system and, more particularly, novel and economical agents for inducing the expression of cytokines by mammalian white blood cells and nutritional supplement compositions containing such stimulators, immunomodulators and agents.

Another object of the present invention is to provide methods for stimulating the expression of cytokines by mammalian white blood cells

The present invention achieves the above-stated objectives by inducing immune responses in mammals which responses involve expression of anti-inflammatory cytokines by the mammalian immune system, more specifically by mammalian peripheral white blood cells, by administering a sugar-processing enzyme to the mammal. Studies by the present inventors show that the sugar-processing enzymes stimulate the mammalian immune system by priming mammalian mononuclear cells to express de novo anti-inflammatory cytokines. Thus, the present invention is considered to enhance the health of the mammalian immune system.

Interferons and Tumor Necrosis Factors (TNFs) function in conjunction with each other to potentiate the immune system. However, the interleukins are the most important cytokines in stimulation of the mammalian immune system. A total of some fifteen cytokines have been described in the literature, the most common among them being IL-1, IL-2, IL-6, IL-10, and IL-12. Those cytokines are among the most preponderant as well. All other interleukins are found in extremely small amounts and, in many cases, are below the detection limit of a particular assay system. In this family of interleukins, some function as pro-inflammatory cytokines. However, the present invention as directed to the induction of anti-inflammatory interleukins especially unregulated interleukins including IL-6, IL-10 and IL-12. In addition to interleukins IL-6, IL-10 and IL-12, other cytokines known to regulate immune-mediated inflammation include IL-5, interferon γ, lymphotoxin and migration inhibition factor (MIF).

The present invention also provides a nutritional supplement containing one or more sugar-processing enzymes and at least one nutritional source such as processed vitamins, minerals, herbs and plant extracts.

The terminology “sugar-processing enzyme,” as used herein, is an enzyme which acts to break down complex sugars or polysaccharides. A polysaccharide is defined as a molecule containing twenty-five or more monosaccharide unites. In contrast, molecules containing 5 to 25 monosaccharide units are described as oligiosaccharides. Thus the enzymes used in the present invention may also be described as “polysaccharide-processing enzymes.”

The term “particulate” as used herein has reference to powders and granules having average particulate sizes of at least 5 microns.

“Cellulase” and “glucolytic enzyme,” as used herein, have the respective, art-recognized meanings previous defined.

“Processed vitamins” is intended to include synthesized vitamins as well as vitamins and vitamin-containing extracts recovered (isolated) from plants or animals.

### BRIEF DESCRIPTION OF THE DRAWINGS

In the drawings:

FIG. 1 is a series of bar graphs of ELISA assay results for IL-1β release by PBMC incubated in the presence of various glucolytic enzymes;

FIG. 2 is a series of bar graphs of ELISA assay results for IL-10 release by PBMC incubated in the presence of various glucolytic enzymes;

FIG. 3 is a series of bar graphs of ELISA assay results for IL-12 release by PBMC incubated in the presence of various glucolytic enzymes;

FIG. 4 is a series of bar graphs of ELISA assay results for IFN-γ release by PBMC incubated in the presence of various glucolytic enzymes;

FIG. 5 is a series of bar graphs of ELISA assay results for TNF-α release by PBMC incubated in the presence of various glucolytic enzymes;

FIG. 6 is a graph of ELISA assay data comparing immuno modulating activities of Econase CEP and Enzoco cellulase FG —as represented by induction of cytokines in cell culture; and

FIG. 7 is a graph of ELISA assay data comparing immuno modulating activities of En and Ec—as represented by induction of cytokines in cell culture.

### DESCRIPTION OF THE PREFERRED EMBODIMENTS

The sugar-processing enzymes preferred for use in the invention are those of plant origin, especially the cellulases and glucolytic enzymes.

Suitable cellulases include:

endoglucanase

exoglucanase

β-glucosidase

Suitable glucolytic enzymes include:

alpha-galactosidase

maltase

Essentially all glucolytic enzymes are derived from plants. Cellulases, on the other hand, are not only derived from plants but may also occur naturally in various organisms, most notably in marine fungi. In fact, one cellulase, Econase, is of marine origin. Both glucolytic enzymes and cellulases, isolated from their sources in purified form, are commercially available in particulate forms such as powders and granules.

The sugar-processing enzymes may be administered to a mammal orally as nutraceuticals and nutritional supplements. These compositions additionally contain ingredients found in conventional food and nutritional supplements, such as one or more of processed vitamins, minerals, herbs and plant extracts.

The recommended daily oral dosage for each individual enzyme is 700 milligrams for an individual human of average size and weight (70 kilograms). In combination with each other, the relative amounts of enzymes may vary such that they deliver a total of 700 milligrams as a recommended dosage. The daily dosage could range from 100 milligrams to 2000 milligrams or, preferably, from 100 milligrams to 1000 milligrams or, more preferably, from 500 milligrams to 750 milligrams.

For topical application and in periodontal products, the daily dosage may be as low as 50 to 100 milligrams, depending upon the intended use. The dosage range for topical applications may vary from 5 milligrams to 100 milligrams.

Induction of Cytokine Release from Peripheral Mononuclear Blood Cells (PBMC) by Glucolytic Enzymes

Peripheral mononuclear/heparinized blood cells (PBHC) were isolated from healthy donor blood samples by density gradient centrifugation. The cells were incubated in cell culture plates (24 wells) in the culture medium described below with 2×10⁶cells were incubated per well with and without the test enzymes (enzyme amounts: 100 μg/ml; 10 μg/ml; 1 μg/ml and 0.1 μg/ml) in a final volume of 3 ml with and without reference stimuli.

RPMI 1640 was used as medium for the cell culture, it contained 2.0/1 NaHCO₃, and were supplemented with the following additives:

| | |
|---|---|
| inactivated fetal calf serum (1 h 56° C.; FCS) | 10% |
| Gentamycin | 100 μg/ml |
| sodium pyruvate | 1 mM |
| N-acetyl-L-alanyl-L-glutamine | 2 mM |

The incubations took place in an incubator at 37° C., in a humidified atmosphere under 5% (vol/vol) CO₂.

The following enzyme preparations were admixed with the cells and culture medium in amounts of 100 μg/ml, 10 μg/ml, 1 μg/ml and 0.1 μg/ml.

I. Econase CEP (EDC, New York/N.Y.) (control)

Lot: S-9930

CEP was used as a reference to compare the effects of the different enzymes.

II. Glucoamylase Concentrate (Valley Research Inc., South Bend/Ind.)

Lot: BT 7122 L

Glucoamylase Concentrate is an exoglucosidase which is characterized by its ability to hydrolyze α-D-1,4-glucosidic linkages in melibiose, raffinose and stachyose espc. The enzyme is a purified food grade enzyme from a selected strain of Aspergillus niger. Glucoamylase Concentrate exhibits a wide temperature optimum between 50° C. and 60° C. and is stable over a pH range of 2.5-6.0 (optimum 3.5-5.0).

III. Maltase (Valley Research Inc., South Bend/Ind.)

Lot: FW 7141 B

Maltase is characterized by its ability to hydrolyze maltose. The enzyme is a purified food grade enzyme from a selected strain of Aspergillus oryzae. Maltase exhibits a wide temperature optimum between 35° and 50° C. and is stable over a pH range of 4.0-6.0.

IV. Validase® AGS 25 Concentrate (Valley Research Inc., South Bend/Ind.)

Lot: CR 8171 D

Validase® AGS 25 Concentrate is an alpha-galactosidase which is characterized by its ability to hydrolyze α-D-1,6-glucosidic branch points and the predominating α-D-1,6-glucosidic linkages of starch. The enzyme is purified food grade enzyme from a selected strain of Aspergillus niger. Vailidase® AGS 25 Concentrate exhibits a wide temperature optimum between 50° and 60° C. and is stable over a pH range of 3.0-9.0 (optimum 4.0-6.5).

V. Validase® FAA Concentrate (Valley Research Inc., South Bend/Ind.)

Lot: AZ 8262 B

Validase® FAA Concentrate is characterized by its ability to hydrolyze dextrin, saccharose and gelatinized starch. The enzyme is a purified food grade enzyme from a selected strain of Aspergillus oryzae. Validase® FAA Concentrate exhibits a wide temperature optimum up to 65° and is stable over a pH range of 2.5-5.5.

VI. Validase® Invertase (Valley Research Inc., South Bend/Ind.)

Lot: HS 8011 E

Validase® Invertase is characterized by its ability to hydrolyze sucrose. The enzyme is a purified food grade enzyme preparation from yeast. Validase® Invertase exhibits a wide temperature optimum between 50° and 60° C. and is stable over a pH range of 2.5-5.5.

The enzymes (100 μg/ml) were also proofed for cytokine induction after inactivation (80° C. for 1 h), and the results evaluated for proof of immunomodulating properties dependency on the enzyme activity.

A mixture of different immunological stimuli was used for the induction of the cytokine release. The mixture contained pokeweed mitogen (PWM), phytohaemagglutinin (PHA), Concanavalin A (ConA) and Lipopolysaccharid (LPS) from E. coli (Serotyp 055:B5) in a final concentration from 1 μg/ml per each component.

Aliquots of the supernatant in each test well were taken after 24 hours and 48 hours. Assays for IL-1β, IL-10, IL-12, IFN-γ and TNF-α were carried out with specific ELISAs (enzyme linked immuno sorbent assay) as follows:

| | |
|---|---|
| Interleukin-1β (IL-1β) | 24 hours |
| Interleukin-12 (IL-12) | 24 hours |
| Tumor necrosis Factor-α (TNF-α) | 24 hours |
| Interferon-γ (IFN-γ) | 48 hours |
| Interleukin-10 (IL-10) | 48 hours |

The supernatants of the cell cultures to be examined were won to the corresponding times, aliquoted and frozen up until the determination at −80° C.

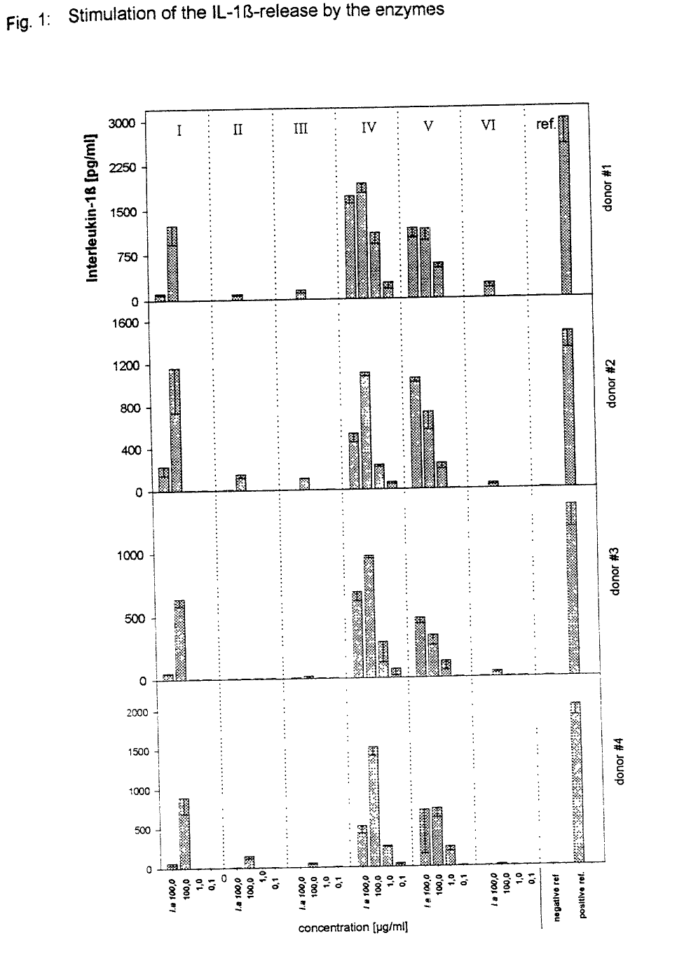

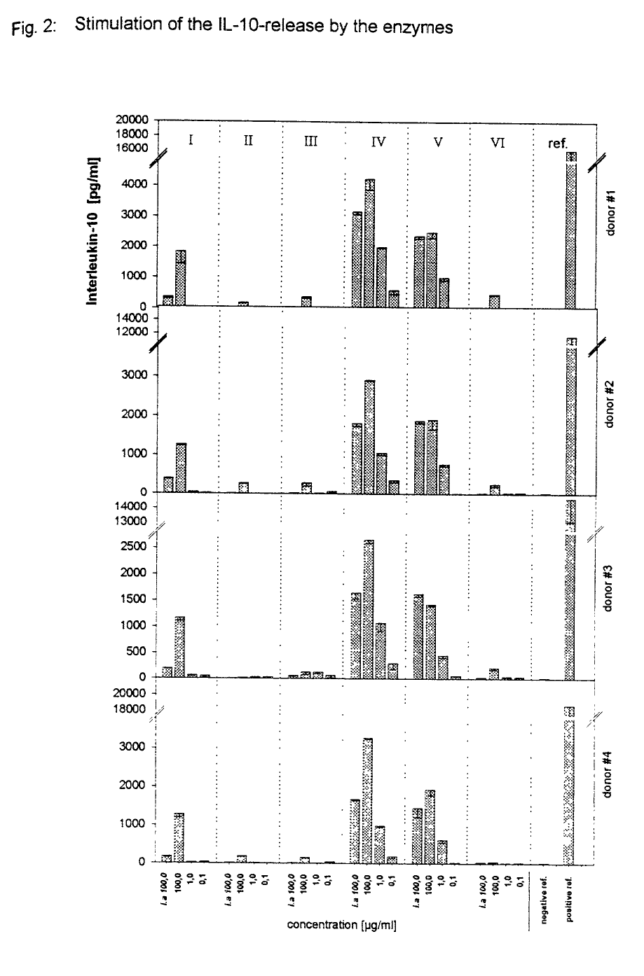

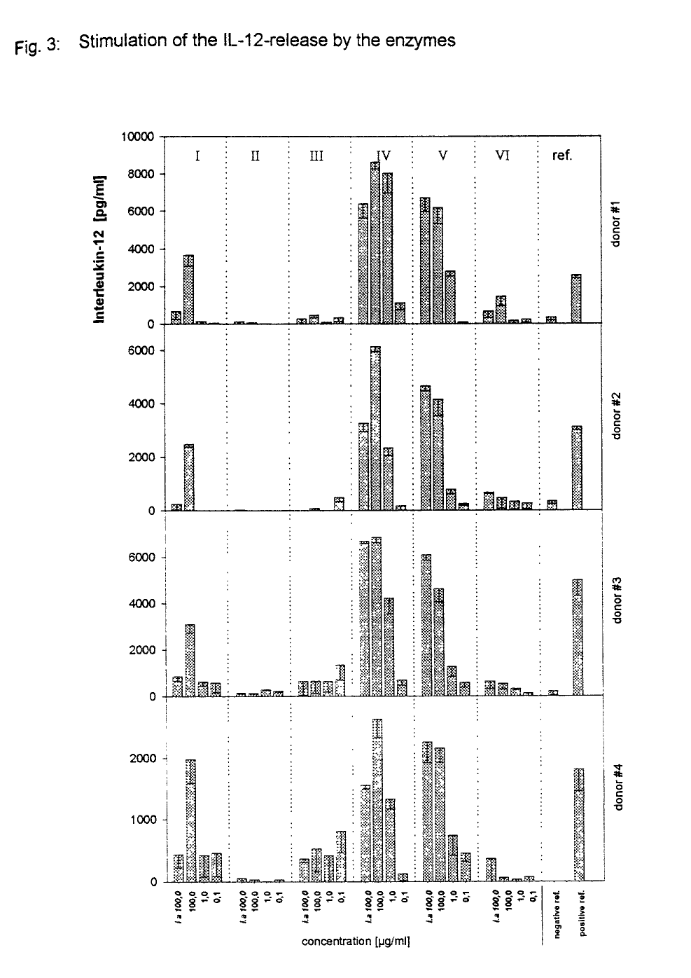

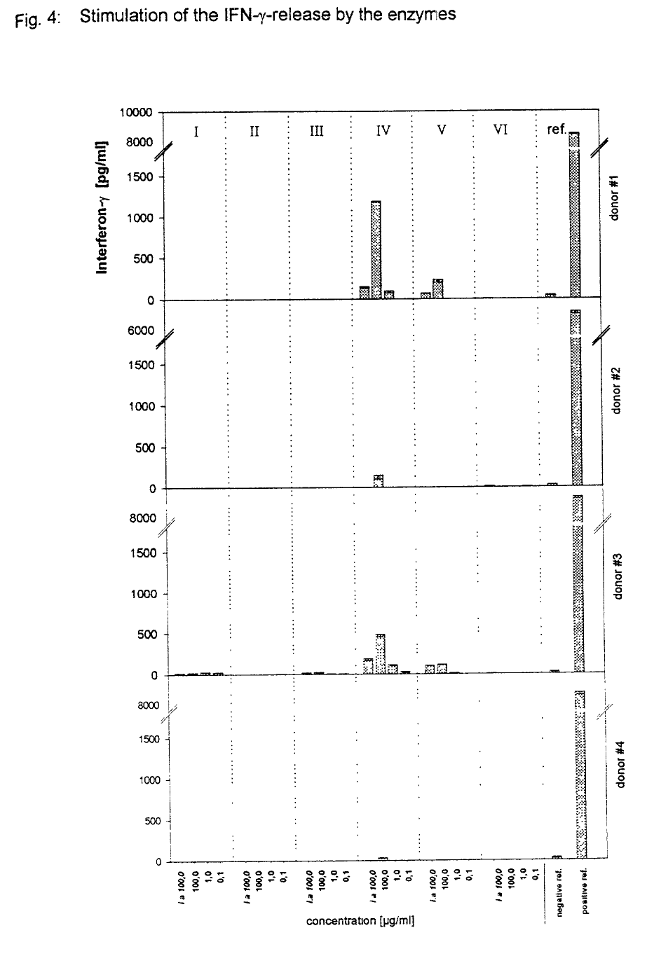

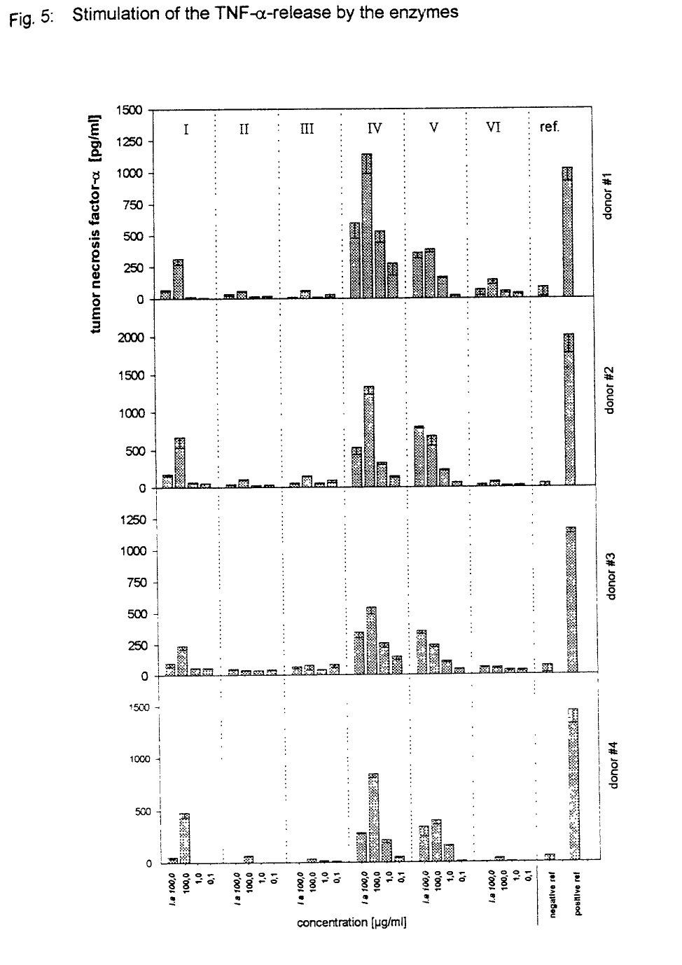

The results of the ELISA assays are shown in FIGS. 1-5.

As expected, the amplitude of cytokine induction varies from reproducible detectable to moderate to extremely high (remarkable).

Econase CEP was used as reference. As expected, its IL-1β signal is low. Glucoamylase Concentrate, Maltase and the Validase® Invertase exhibit low inductive potential for IL-1β. In contrast to that the amount of IL-1β, measured after stimulation by Validase® AGS 25 Concentrate, is remarkably high. Furthermore, the induction by Validase® FAA Concentrate differs from moderate to high.

The IL-10 signal is induced by the reference signal. Econase CEP induces moderately (100 μg/ml), while those of Glucoamylase Concentrate, Maltase and the Validase® AGS 25 Concentrate, are remarkably high. The signal induced by the mixture of stimuli of the positive control is much higher for a singular stimulus than the signal induced by Validase® AGS 25. Furthermore, the induction by Validase® FAA Concentrate is moderate, but clearly dose-dependent.

Econase CEP induces a moderate IL-12 signal, but a high enzyme concentration (100 μg/ml) is required, while Glucoamylase Concentrate, Maltase and the Validase® Invertase exhibit low inductive potential. In contrast to that the IL-12 amounts, measured after stimulation by Validase® AGS 25 Concentrate and Validase® FAA Concentrate, are remarkably high.

Neither Econase CEP nor the enzymes Glucoamylase Concentrate, Maltase, Validase® Invertase and Validase® FAA Concentrate are capable of inducing a relevant IFN-γ signal. Validase® AGS 25 Concentrate can activate the cells of 2 from 4 donors to produce moderate IFN-γ amounts.

Econase CEP and Validase® FAA Concentrate stimulate moderate TNF-α signals, while Glucoamylase Concentrate, Maltase and the Validase® Invertase exhibit, if any, only a very low inductive potential. In contrast the TNF-α amount, as measured after stimulation by Validase® AGS 25 Concentrate, is high, even in comparison to the other enzymes and the positive control.

Induction of Cytokine Release from Mononuclear White Blood Cells (PBMC) by Cellulases

Heparinized blood from healthy donors was used to isolate peripheral monocytes (PBMC) by density gradient centrifugation. Each experimental data point utilized 2×10⁶ cells under standard cell culture conditions with and without cellulase (0.1-100 μg/ml). Commercially available purified cellulase from Penicillium funiculosum (Sigma), Econase CEP (hereinafter “Ec”; Enzyme Development Corporation) and Enzeco cellulase FG concentrate (hereinafter “En”; Enzyme Development Corporation) were the cellulases used in the study.

RPMI 1640 was used as medium for the cell culture, it contained 2.0 g/l NaHCO₃, and were supplemented with following additives:

| | |
|---|---|
| inactivated fetal calf serum (1 h 56° C.; FCS) | 10% |
| Gentamycin | 100 μg/ml |
| sodium pyruvate | 1 mM |
| N-acetyl-L-alanyl-L-glutamine | 2 mM |

The incubation took place in the incubator at 37° C., in a humidified atmosphere and 5% (vol/vol) CO₂. The cells of 5 donors were taken to investigate En and Ec. The reference cellulase (Sigma) was tested with cells of 3 donors.

The cells were incubated in cell culture plates (24 wells) with the media and conditions described above. 2×10⁶ cells were incubated per well, with and without the test compounds (100 μg/ml; 10 μg/ml and 0.1 μg/ml) in a final volume of 3 ml and with and without the stimuli.

A mixture of different immunological stimuli was used for the induction of the cytokine release. The mixture contained pokeweed mitogen (PWM), phytohaemagglutinin (PHA), Concanavalin A (ConA) and Lipopolysaccharid (LPS) from E. coli (Serotyp 055:B5) in a final concentration from 1 μg/ml per each component.

Aliquots of the supernatant were taken after 24 hours and 48 hours. The determinations were carried out with specific ELISAs (enzyme linked immuno sorbent assay) as follows:

| | |
|---|---|
| Interleukin-1β (IL-1β) | 24th hour |
| Interleukin-10 (IL-10) | 24th hour |
| Interleukin-12 (IL-12) | 48th hour |
| Interferon-γ (IFN-γ) | 48th hour |
| Tumor Necrosis Factor-α (TNF-α) | 24th hour |

The supernatants of the cell culture to be examined were won to the corresponding times, aliquoted and frozen up until the determination at −80° C.

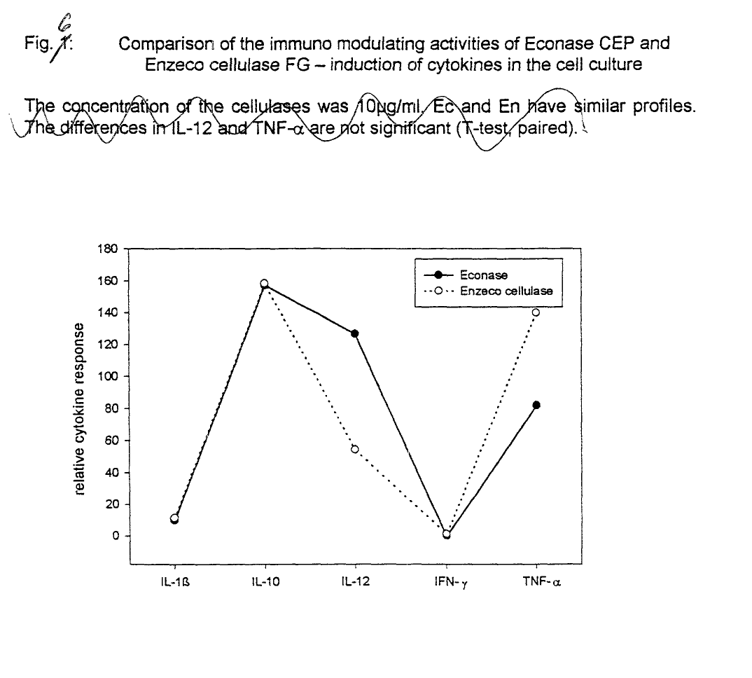

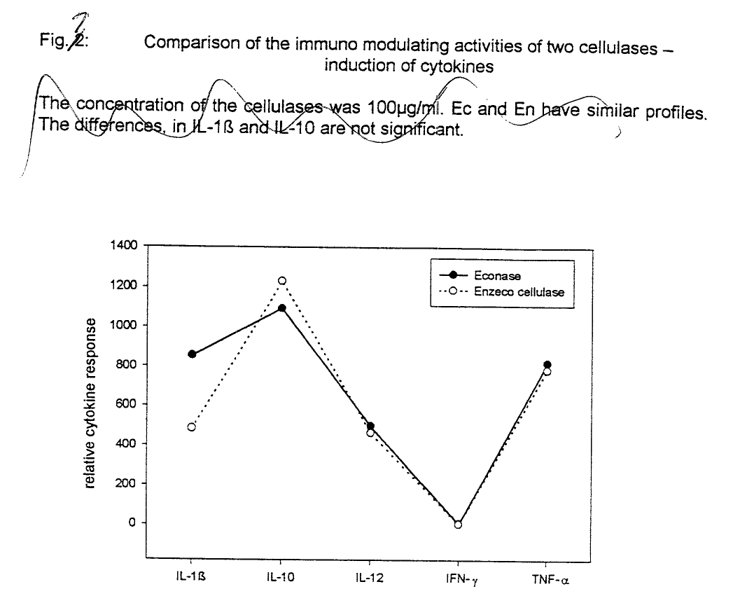

The results of the assays are shown in FIGS. 6 and 7. As seen FIGS. 6 and 7, Ec and En activate human immuno competent cells to produce the cytokines IL-1μ, IL-10, IL-12, and TNF-α. The T-cell cytokine IFN-γ probably cannot be stimulated.

The IL-12 and TNF-α signals induced by Ec and En do not differ significantly (t-Test; p=0.108 and p=0.537 resp.)

The cytokine signals of Ec and En are very similar. It can be concluded that the immno modulating activities come from the cellulases and not from contaminations of the enzymes such as peptides, glucans etc.

The cellulases were tested over a wide range of concentrations, from 1 ng-100 μg/ml, and in comparison to a cellulase derived from the fungus Penicillium funicolosum (Sigma Inc.). This latter cellulase product also gave significant cytokine signals in lower concentrations and the response follows more or less a classical dose effect kinetic.

Both En and Ec cellulases were found to be strong inducers of IL-10. This anti-inflammatory cytokine is an important down regulating immune molecule of inflammatory processes. In vivo IL-10 is involved in the down regulation of pro-inflammatory cytokines IK-1β, IL-6 and TNF-α. These cytokines play a key role in perpetuating autoimmune diseases like rheumatic arthritis and chronic inflammation.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

We claim:

1. A method of stimulating the expression of anti-inflammatory cytokines by mammalian peripheral white blood cells comprising administering a cytokine expression enhancing amount of a sugar-processing enzyme to a mammal.

2. A method according to claim 1 wherein said sugar-processing enzyme is a glucolytic enzyme.

3. A method according to claim 1 wherein said sugar-processing enzyme is a cellulase.

4. A method according to claim 1 wherein said sugar-processing enzyme is alpha-galactosidase.

5. A nutritional supplement comprising a particulate sugar-processing enzyme and at least one member selected from the group consisting of processed vitamins, minerals, herbs and plant extracts.

6. A nutritional supplement according to claim 5 wherein said enzyme is a cellulase.

7. A nutritional supplement according to claim 5 wherein said enzyme is a glucolytic enzyme.

8. A nutritional supplement according to claim 5 wherein said enzyme is alpha-galactosidase.
